(19) United States
(12) Patent Application Publication
(10) Pub. No.: US 2006/0149365 A1
(43) Pub. Date: Jul. 6, 2006

(54) Stent with eccentric coating

(75) Inventors: Daniel Fifer, Windsor, CA (US); John Nolting, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., San Rosa, CA (US)

(21) Appl. No.: 10/531,694

(22) PCT Filed: Oct. 21, 2003
(86) PCT No.: PCT/US03/33525

§ 371 Date: Oct. 20, 2005

Related U.S. Application Data
(60) Provisional application No. 60/420,773, filed on Oct. 22, 2002.

(51) Int. Cl. A61F 2/06

(52) U.S. Cl. 623/1.46

(57) ABSTRACT

The stent (120) with an eccentric coating (130) of the present invention provides a coating having a different coating thickness on the stent outer diameter and stent inner diameter, i.e., an eccentric coating. The eccentric coating can be the primary carrier for a drug or other therapeutic agent. The eccentric coating can be thicker on the stent outer diameter to supply more drug to the vessel wall in which the coated stent is deployed and less drug to the vessel lumen. In one embodiment, a cap coating (125) can be disposed on the eccentric coating to protect the eccentric coating, control the elution rate from the eccentric coating, provide an additional drug carrier, or provide combinations thereof. The eccentric coating can be applied by spraying a coating liquid on the stent outer diameter with a fixture mandrel interior to the stent to regulate the spray to the stent inner diameter.

### RELATED APPLICATIONS

This application is a continuation-in-part of pending United States Provisional Patent Application No. 60/420,773, filed Oct. 22, 2002, the priority of which is hereby claimed.

### TECHNICAL FIELD

The technical field of this disclosure is medical implant devices, particularly, coated stents.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion comprised of, for example, a plaque or thrombus. After inflation, the pressurized balloon exerts a compressive force on the lesion thereby increasing the inner diameter of the affected vessel. The increased interior vessel diameter facilitates improved blood flow. Soon after the procedure, however, in a significant number of cases treated vessels re-narrow.

To reduce occurrence of restenosis, short flexible cylinders, or stents, constructed of metal or various polymers are implanted within the vessel to maintain lumen size. The stents act as a scaffold to support the lumen in an open position. Various configurations of stents include a cylindrical tube defined by a mesh interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when the stents are deployed. Stents can also be self-expanding, growing to a final diameter when deployed without mechanical assistance from a balloon or like device.

Stents have been used with coatings to deliver drug or other therapy at the site of the stent. The coating may also be passive. The coating can be applied as a liquid containing the drug or other therapeutic agent dispersed in a polymer/solvent matrix. The liquid coating then dries to a solid coating upon the stent. The liquid coating can be applied by dipping or spraying the stent while spinning or shaking the stent to achieve a uniform coating. Combinations of the various application techniques can also be used.

The purpose of the coating is to deliver the drug to the tissue adjacent to the stent, such as the interior wall of an artery or vessel. Therefore, it is most important that the drug be present on at least the outer diameter of the stent. The coating has generally been applied uniformly over the inside and outside diameters of the stent, in one or more layers over the stent wires. Because drugs to reduce the occurrence of restenosis are only required at the stent outer diameter where the stent contacts the vessel tissue and not at the stent inner diameter containing blood, applying a drug-containing coating uniformly can lead to adverse drug effects or delivery to non-target tissue. For example, a drug on the inner diameter can flow into the blood where it is not beneficial. In addition, some drugs and polymers require a certain quantity or concentration before an effective therapy can be delivered. A uniform coating of a thickness necessary to provide sufficient drug at the outer diameter of the stent to constitute an effective therapy may interfere with blood flow through the inner lumen of the stent.

It would be desirable to have a stent having an eccentric coating that would overcome the above disadvantages.

### SUMMARY OF THE INVENTION

One aspect of the present invention provides a stent having an eccentric coating to target drug delivery to tissue at the outer diameter of the stent

Another aspect of the present invention provides a stent having an eccentric coating to avoid drug waste.

Another aspect of the present invention provides a stent having an eccentric coating to reduce drug delivery to the blood.

Another aspect of the present invention provides a stent having an eccentric coating able to provide different characteristics and therapies at the vessel wall and lumen sides.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 shows a stent delivery system made in accordance with the present invention.

FIGS. 2 & 3 show a stent and a cross section, respectively, of a coated stent made in accordance with the present invention.

FIGS. 4A, 4B, 4C and 4D show various cross-sections of stent wires with eccentric coatings made in accordance with the present invention.

FIG. 5 shows a method of manufacturing a coated stent made in accordance with the present invention.

FIGS. 6-8 show a method of manufacturing a stent made in accordance with the present invention using a fixture mandrel to regulate flow.

FIG. 9 shows an alternate embodiment of a method of applying a coating to a stent.

FIG. 10 shows yet another alternate embodiment of a method of applying a coating to a stent.

### DETAILED DESCRIPTION OF THE INVENTION

The stent with an eccentric coating of the present invention provides a coating having a different coating thickness on the stent outer diameter and stent inner diameter, i.e., an eccentric coating. The eccentric coating can be the primary carrier for a drug or other therapeutic agent. The eccentric coating can be thicker on the stent outer diameter to supply more drug to the vessel wall in which the coated stent is deployed and less drug to the vessel lumen. In one embodiment, a cap coating can be disposed on the eccentric coating to protect the eccentric coating, control the elution rate from the eccentric coating, provide an additional drug carrier, or provide combinations thereof. The eccentric coating can be applied by spraying a coating liquid on the stent outer diameter with a fixture mandrel interior to the stent to regulate the spray to the stent inner diameter.

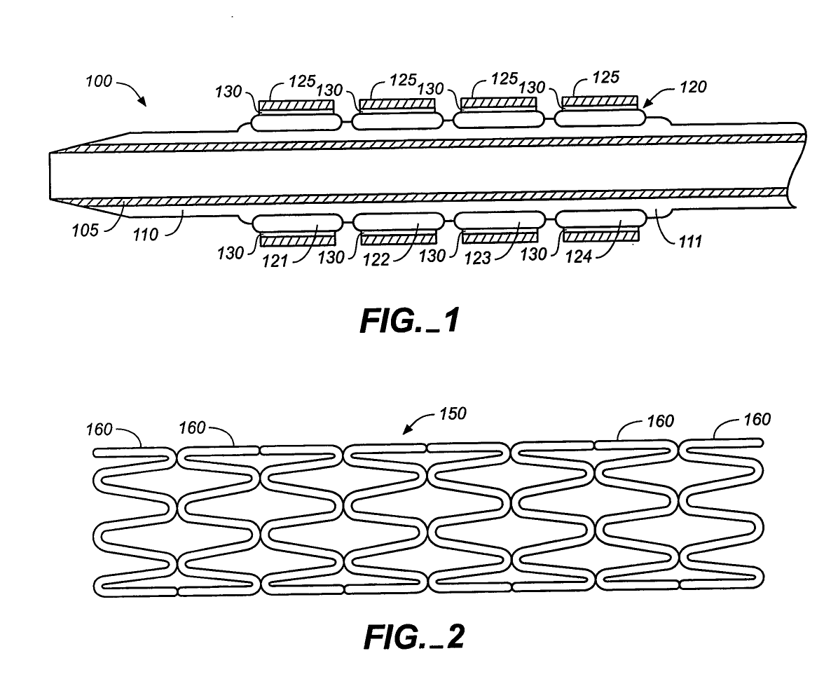

FIG. 1 shows a stent delivery system made in accordance with the present invention. The stent delivery system 100 includes a catheter 105, a balloon 110 operably attached to the catheter 105, and a stent 120 disposed on the balloon 110. The balloon 110, shown in a collapsed state, may be any variety of balloons capable of expanding the stent 120. The balloon 110 may be manufactured from any sufficiently elastic material such as polyethylene, polyethylene terephthalate (PET), nylon, or the like. In one embodiment, the balloon 110 may include retention means 111, such as mechanical or adhesive structures, for retaining the stent 120 until it is deployed. The catheter 105 may be any variety of balloon catheters, such as a PTCA (percutaneous transluminal coronary angioplasty) balloon catheter, capable of supporting a balloon during angioplasty.

The stent 120 may be any variety of implantable prosthetic devices capable of carrying a coating known in the art. In one embodiment, the stent 120 may have a plurality of identical cylindrical stent segments placed end to end. Four stent segments 121, 122, 123, and 124 are shown, and it will be recognized by those skilled in the art that an alternate number of stent segments may be used. The stent 120 includes at least one cap coating 125, which can be applied to the stent 120 by dipping or spraying the stent 120 with a coating liquid, or applying the coating liquid with a combination of methods. The cap coating 125 can be applied as a liquid polymer/solvent matrix. A therapeutic agent can be incorporated in the cap coating 125, or can be omitted and the cap coating 125 included for its mechanical properties alone.

An eccentric coating 130 between the cap coating 125 and the stent 120 is the primary carrier for a therapeutic agent. The cap coating 125 can be applied as a liquid containing the drug or other therapeutic agent dispersed in a polymer/solvent matrix.

The cap coating 125 and eccentric coating 130 are merely exemplary, and it should be recognized that other coating configurations, such as multiple coating layers, are possible. Although the cap coating 125 and the eccentric coating 130 are shown schematically on the outer circumference of the stent 120, the cap coating 125 and the eccentric coating 130 can coat the whole stent 120, both inside and outside, and around the cross section of individual stent wires. In another embodiment, the eccentric coating 130 can be present on a portion of the stent 120 without a cap coating 125 on that same portion.

The cap coating 125 and eccentric coating 130 can be a polymer including, but not limited to, urethane, polyester, epoxy, polycaprolactone (PCL), polymethylmethacrylate (PMMA), PEVA, PBMA, PHEMA, PEVAc, PVAc, Poly N-Vinyl pyrrolidone, Poly (ethylene-vinyl alcohol), combinations of the above, and the like. Suitable solvents that can be used to form the liquid coating include, but are not limited to, acetone, ethyl acetate, tetrahydrofuran (THF), chloroform, N-methylpyrrolidone (NMP), combinations of the above, and the like. Suitable therapeutic agents include, but are not limited to, antiangiogenesis agents, antiendothelin agents, antimitogenic factors, antioxidants, antiplatelet agents, antiproliferative agents, antisense oligonucleotides, antithrombogenic agents, calcium channel blockers, clot dissolving enzymes, growth factors, growth factor inhibitors, nitrates, nitric oxide releasing agents, vasodilators, virus-mediated gene transfer agents, agents having a desirable therapeutic application, combinations of the above, and the like. Specific example of therapeutic agents include abciximab, angiopeptin, colchicine, eptifibatide, heparin, hirudin, lovastatin, methotrexate, rapamycin, streptokinase, taxol, ticlopidine, tissue plasminogen activator, trapidil, urokinase, and growth factors VEGF, TGF-beta, IGF, PDGF, and FGF.

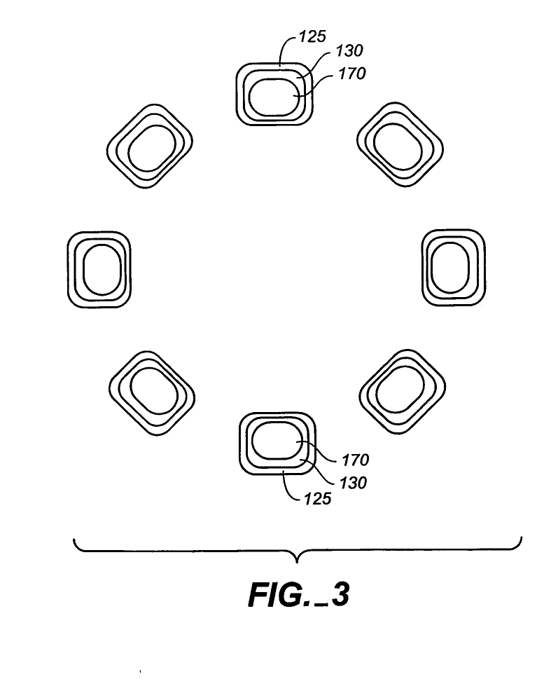

FIG. 2 shows a coated stent made in accordance with the present invention. The stent 150 comprises a number of segments 160. The pattern of the segments 160 can be W-shaped or can be a more complex shape with the elements of one segment continuing into the adjacent segment. The stent 150 can be installed in the stent delivery system of FIG. 1 for implantation in a body lumen.

Referring to FIG. 2, the stent 150 is conventional to stents generally and can be made of a wide variety of medical implantable materials, such as stainless steel (particularly 316-L stainless steel or 316LS), nitinol, tantalum, ceramic, nickel, titanium, aluminum, polymeric materials, tantalum, MP35N, titanium ASTM F63-83 Grade 1, niobium, high carat gold K 19-22, and combinations thereof. The stent 150 can be formed through various methods as well. The stent 150 can be welded, laser cut, molded, or consist of filaments or fibers which are wound or braided together in order to form a continuous structure. Depending on the material, the stent can be self-expanding, or be expanded by a balloon or some other device. The cap coating and eccentric coating can be on the surface of the segments 160.

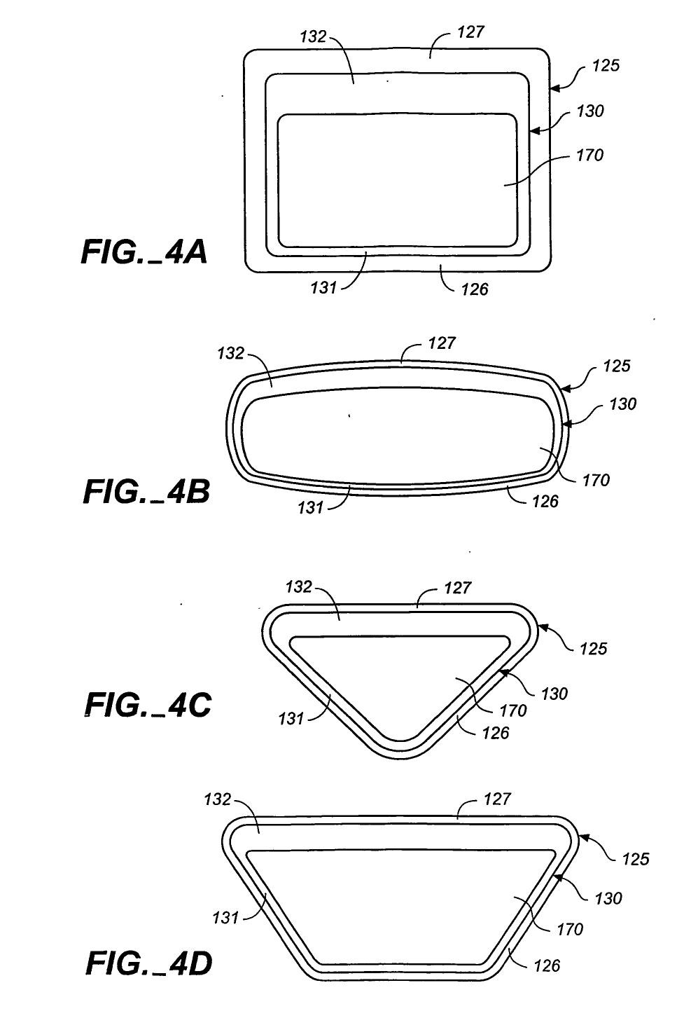

FIG. 3 shows a cross section of a coated stent made in accordance with the present invention. A plurality of stent wires 170 are provided with a cap coating 125 and eccentric coating 130. The stent wires form the segments, which form the stent. Although the cross section of the stent wires 170 is shown as generally rectangular with rounded corners, the cross section can be any number of shapes, for example and without limitation, those shown in FIGS. 4A, 4B, 4C and 4D, depending on fabrication methods, materials, and desired effect.

FIGS. 4A, 4B, 4C and 4D, in which like elements share like reference numbers with FIG. 3, show examples of cross sections of stent wires with eccentric coatings made in accordance with the present invention. The cross sections can be of any number of other shapes depending on fabrication methods, materials, and desired effect. Please note, this is a representation only of the relative eccentricity of the stent coating—the actual stent cross section will vary depending on the type of stent used. An eccentric coating 130 is disposed on the stent wire 170 and a cap coating 125 can be disposed on the eccentric coating 130. The eccentric coating 130 can comprise a first eccentric portion 131 positioned on the surface of the stent directly defining the inner lumen of the stent, i.e disposed towards the inner diameter of the stent and a second eccentric portion 132 positioned on the outer surface of the stent, i.e. disposed towards the outer diameter of the stent. The cap coating 125 can comprise a first cap portion 126 towards the stent inner diameter and a second cap portion 127 towards the stent outer diameter. In an alternate embodiment, the cap coating can be omitted and the eccentric coating provided as the outer layer of the stent.

The eccentric coating 130 can be the primary carrier of the therapeutic agent or drug. To concentrate the therapeutic agent at the vessel wall, rather than the vessel lumen, the second eccentric portion 132 can be thicker and have a greater volume than the first eccentric portion 131. In an alternate embodiment, the second eccentric portion 132 can be thinner and have a lesser volume than the first eccentric portion 131 to concentrate the therapeutic agent at the vessel lumen for dispersion in the blood.

The cap coating 125 can be used for a number of purposes, including, but not limited to, a diffusion barrier to control the elution rate of the therapeutic agent from the eccentric coating 130; a protective barrier to prevent damage to the eccentric coating 130; a drug carrier for the same drug as the eccentric coating 130 or a different drug; a lubricating layer to reduce friction, or a binding layer to increase friction, between the stent and the balloon of the stent delivery system; or various combinations thereof. In one embodiment, the cap coating 125 can be of a single material and uniform thickness to form a concentric cap coating. Such a uniform coating can be produced by dipping the stent in a liquid coating. In another embodiment, the first cap portion 126 and second cap portion 127 can be of different thicknesses or of different materials to provide a number of useful combinations. For example, the second cap portion 127 can be thicker than the first cap portion 126, paralleling the relative thickness of the second eccentric portion 132 and the first eccentric portion 131, respectively. In another example, the second cap portion 127 can be rapidly biodegradable, while the first cap portion 126 is non-biodegradable, so that the vessel wall is exposed to the second eccentric portion 132 and the drug eluting therefrom, but the first cap portion 126 limits drug elution from the first eccentric portion 131 into the vessel lumen. In another example, the second cap portion 127 can be made of one polymer-drug combination of benefit to and compatible with the vessel wall and the first cap portion 126 made of a different polymer-drug combination beneficial to and compatible with the blood stream. In another example, the second eccentric portion 132 is of greater thickness than the first eccentric portion 131, and the first cap portion 126 is of greater thickness than the second cap portion 127, allowing for greater release of drug to the vessel wall while inhibiting drug release into the vessel lumen. Those skilled in the art will appreciate that many useful combinations of the first cap portion 126 and the second cap portion 127, not limited to the examples presented herein, are possible. Additionally, in cases where the cap coating 125 incorporates a drug, a protective, or “sacrificial,” biodegradable coating may be applied over the cap coating 125 to protect the cap coating 125 and the underlying eccentric coating 130 from damage during handling or deployment of the stent.

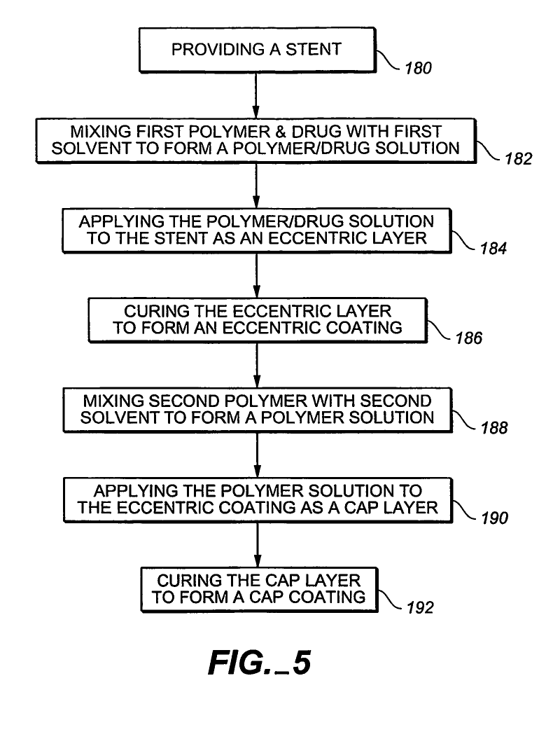

FIG. 5 shows a method of manufacturing a coated stent made in accordance with the present invention. At 180, a stent is provided. A first polymer and drug (or other therapeutic agent) are mixed with a first solvent to form a polymer/drug solution 182. The polymer/drug solution is applied to the stent in an eccentric layer 184 and the eccentric layer cured to form an eccentric coating 186. A second polymer is mixed with a second solvent to form a polymer solution 188. The polymer solution is applied to the eccentric coating in a cap layer 190 and the cap layer cured to form a cap coating 192. With reference to FIG. 5, the use of the terms “polymer,” “solvent” and “drug” in the singular shall, in each case, include them in the plural (i.e., mixtures of one or more polymers, solvents and/or drugs).

Those skilled in the art will appreciate that the method of manufacturing can be varied for the materials used and the results desired. For certain polymer/drug solutions and polymer solutions, the curing step can be omitted or can be a simple drying process. In another embodiment, the first polymer and first solvent can be the same combination as the second polymer and second solvent. In yet another embodiment, the polymer solution can also contain a drug or other therapeutic agent.

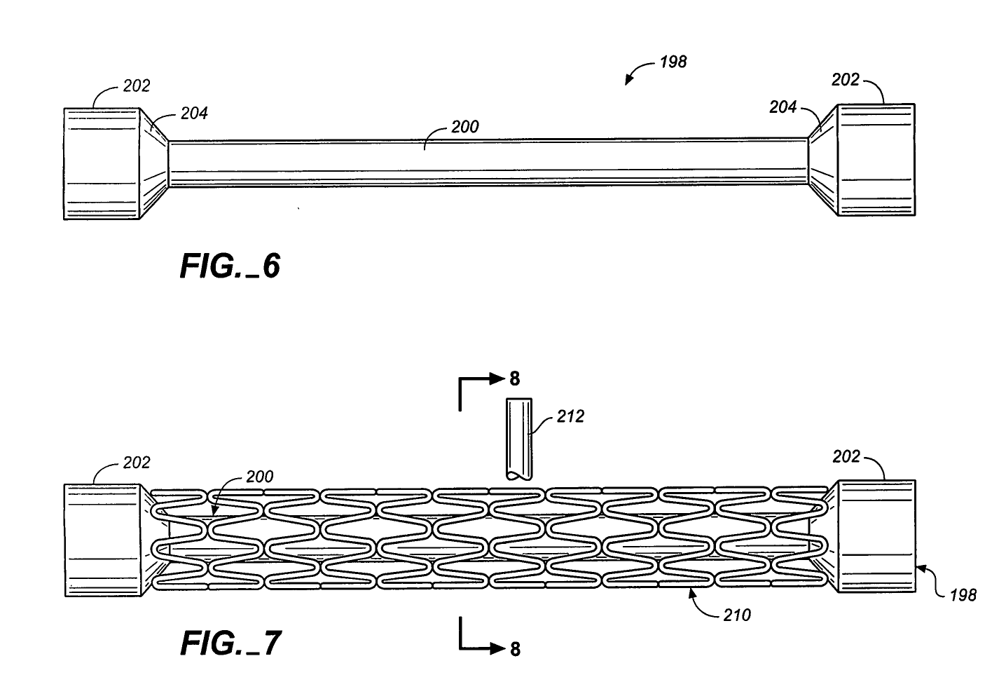

FIGS. 6-8 show a method of manufacturing a coated stent made in accordance with the present invention using a fixture mandrel to regulate flow. Referring to FIG. 6, a coating fixture 198 holds a stent while the coating is applied. The coating fixture 198 comprises a fixture mandrel 200 held in place by retainers 202. The sloping face 204 helps to center and secure the stent during the coating operation. At least one of the retainers 202 can be attached to a drive (not shown) to rotate the stent in a single direction, or back and forth, during the coating operation. The drive can also provide axial movement if desired.

The fixture mandrel 200 can be held between the sloping faces 204 of the retainers 202, or can be fixed to the retainers 202 by threads or other means. At least one of the retainers 202 can be detachable from the retainers 202 to a low installation of the stent to be coated between the retainers 202. The fixture mandrel 200 can be metal or other material to block or redirect spray directed at the stent. The fixture mandrel 200 can be perforated to allow a portion of the spray to pass through the fixture mandrel 200. The fixture mandrel 200 can be made of a sponge-like material or have a sponge-like outer coating to retain spray directed at the stent.

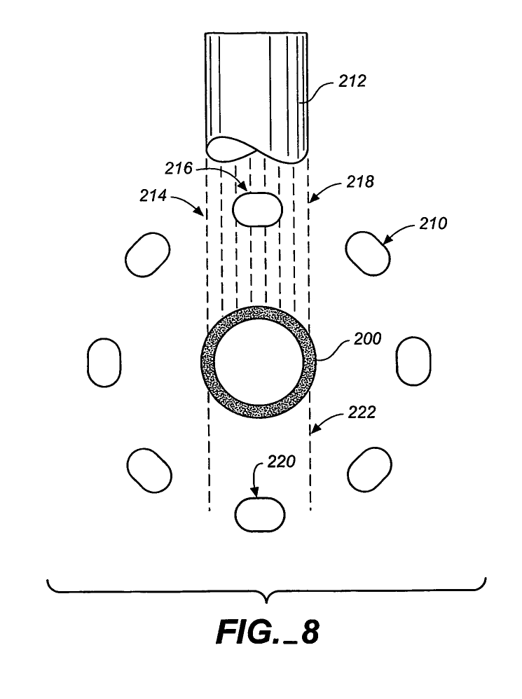

FIG. 7, in which like elements share like reference numbers with FIG. 6, shows a stent mounted on the coating fixture. The coating fixture 198 holds a stent 210 for application of the coating from spray nozzle 212. To apply the coating over the whole stent 210, the spray nozzle 212 can be attached to a drive (not shown) to move the spray nozzle 212 relative to the coating fixture 198. The spray rate from the spray nozzle 212, the movement of the spray nozzle 212, and movement of the coating fixture 198 can be controlled by computerized numerically controlled machines to control the coating pattern and thickness on the stent 210. The stent 210 can be a bare frame of metal or other stent base materials, or can be a stent with one or more coatings previously applied. Alternatively, the stent 210 and coating fixture 198 can move relative to a stationary spray nozzle 212, or both the stent 210 coating fixture 198 and the spray nozzle 212 can move relative to one another.

FIG. 8, in which like elements share like reference numbers with FIG. 6 & 7, shows a cross section of a stent mounted on the coating fixture during coating application. The spray nozzle 212 directs a spray 214 toward the stent 210. The stent outer diameter 216 receives the full spray 218. The fixture mandrel 200, as well as the struts or wires of the stent 210 themselves, blocks a portion of the spray, so that the stent inner diameter 220 receives a shadow spray 222. The spray rate difference between the full spray 218 and the shadow spray 222 results in a thicker coating on the stent outer diameter 216 than the stent inner diameter 220, i.e., an eccentric coating. The stent 210 can be rotated in a single direction or in alternating directions to achieve coating characteristics as desired, depending on the coating materials used. The fixture mandrel 200 can optionally rotate with the stent.

The fixture mandrel 200 is shown as circular for purposes of illustration, but can be other shapes as desired. The fixture mandrel 200 can be perforated to allow spray to pass through the fixture mandrel 200 and reduce the spray rate difference between the full spray 218 and the shadow spray 222. In another embodiment, the fixture mandrel 200 can be made of or coated with a sponge-like material to partially retain the spray and to reduce splatter from the surface of the fixture mandrel 200. In yet another embodiment, the cross section of the fixture mandrel 200 can vary axially to vary the coating eccentricity of the coating with axial position. For example, the middle of the fixture mandrel can have a larger cross section to make the coating more eccentric (more coating on the stent outer diameter) at the middle of the stent, allowing a greater drug loading in the middle. Similarly, the ends of the fixture mandrel can be wider to make the coating more eccentric at the ends of the stent. In other embodiments a specific, uniform degree of eccentricity of the coating along the longitudinal axis of the stent can be determined by setting a specific, uniform cross section of the fixture mandrel.

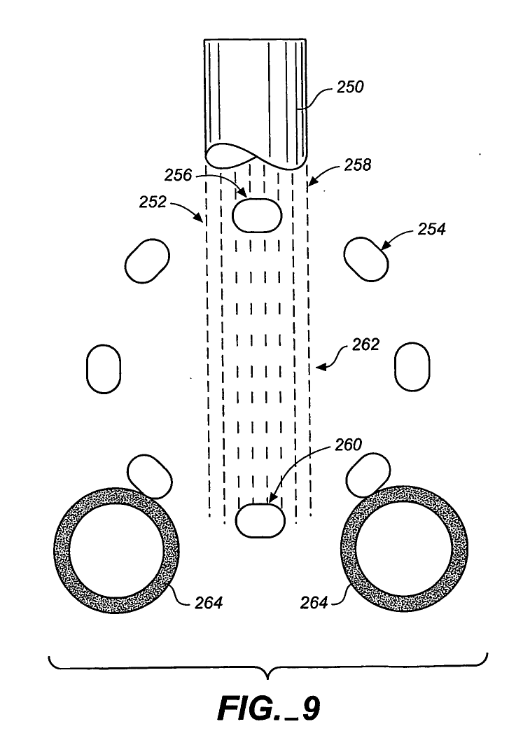

FIG. 9 shows an alternate embodiment of a method of applying a coating to a stent. The spray nozzle 250 directs a spray 252 toward the stent 254. The stent 254 can be a bare frame of metal or other stent base materials, or can be a stent with one or more coatings previously applied. The stent outer diameter 256 receives the full spray 258. The stent outer diameter 256 blocks a portion of the spray, so that the stent inner diameter 260 receives a shadow spray 262. The spray rate difference between the full spray 258 and the shadow spray 262 depends on the geometry between the spray nozzle 250 and the stent outer diameter 256, including the proximity of the spray nozzle to the outside diameter and the size of the spray nozzle aperture.

In one embodiment, where the stent outer diameter 256 blocks a substantial portion of the spray, the spray rate difference between the full spray 258 and the shadow spray 262 results in a thicker coating on the stent outer diameter 216 than the stent inner diameter 220, i.e., an eccentric coating. In another embodiment, where the stent outer diameter 256 blocks little of the spray, the spray rate difference between the full spray 258 and the shadow spray 262 results in substantially equal coating thicknesses on the stent outer diameter 216 and the stent inner diameter 220, i.e., a uniform coating. The stent 210 can be rotated by fixture rollers 264 in a single direction or in alternating directions to achieve coating characteristics as desired, depending on the coating materials used. The fixture rollers 264 can be used alone or can be used in conjunction with the retainers described in FIG. 7.

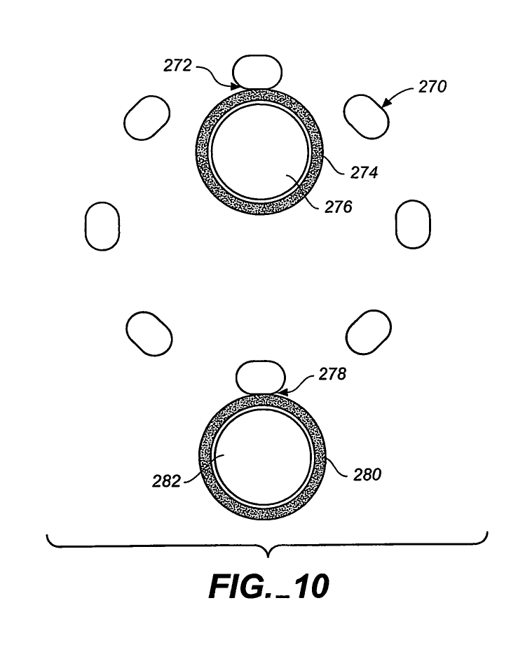

FIG. 10 shows yet another alternate embodiment of a method of applying a coating to a stent. A pad carrying the liquid coating and disposed about a transfer roller transfers the liquid coating to the stent 270. The stent 270 can be a bare frame of metal or other stent base materials, or can be a stent with one or more coatings previously applied. To coat the stent inner diameter 272, inner pad 274 disposed about inner transfer roller 276 rotates against the inner diameter of the stent 270. Typically, the inner transfer roller 276 and stent 270 can rotate in the same direction, but for certain stent and pad finishes, and liquid coating consistencies, the transfer roller 276 and stent 270 can rotate in opposite directions. To coat the stent outer diameter 278, the outer pad 280 disposed about outer transfer roller 282 rotates against the outer diameter of the stent 270. Typically, the outer transfer roller 282 and stent 270 can rotate in the opposite directions, but for certain stent and pad finishes, and liquid coating consistencies, the transfer roller 276 and stent 270 can rotate in opposite directions.

Although FIG. 10 provides the example of applying the liquid coating to the stent inner and outer diameters simultaneously, the stent inner diameter and stent outer diameter could be coated in different steps. For example, the stent outer diameter can be coated to one thickness with one liquid coating, cured as necessary, and then the stent inner diameter coated with a different liquid coating to a different thickness. In another embodiment, the stent outer diameter could be coated with a biodegradable coating and the stent inner diameter could be coated with a non-biodegradable coating. This could allow the cap coating near the vessel wall to degrade quickly to permit underlying therapeutic agents to work at the vessel wall, while reducing the therapeutic agents entering the blood. Those skilled in the art will appreciate that many combinations of stent inner and outer diameter coating thicknesses, therapeutic agents, and coating materials can be used to advantage as desired.

It is important to note that FIGS. 1-10 illustrate specific applications and embodiments of the present invention, and is not intended to limit the scope of the present disclosure or claims to that which is presented therein. For example, the cap coating and eccentric coating can be applied in a variety of conventional ways, including painting, spraying, dipping, wiping, electrostatic deposition, vapor deposition, epitaxial growth, combinations thereof, and other methods known to those of ordinary skill in the art. The means of applying the liquid coating, such as spray nozzles or pads, can be moved in various paths relative to the stent to achieve particular patterns and thickness variations. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and a IU changes that come within the meaning and range of equivalents are intended to be embraced therein.

1. A coated stent comprising:
a stent, the stent having a stent inner diameter and a stent outer diameter; and
an eccentric coating, the eccentric coating having a first eccentric portion and a second eccentric portion, the first eccentric portion disposed on the stent inner diameter and the second eccentric portion disposed on the stent outer diameter;
wherein the first eccentric portion and the second eccentric portion have different thicknesses.

2. The coated stent of claim 1 wherein the second eccentric portion is thicker than the first eccentric portion.

3. The coated stent of claim 1 wherein the first eccentric portion is thicker than the second eccentric portion.

4. The coated stent of claim 1 wherein the eccentric coating includes a therapeutic agent.

5. The coated stent of claim 4 further comprising a cap coating disposed on the eccentric coating, the cap coating regulating elution of the therapeutic agent from the eccentric coating.

6. The coated stent of claim 1 further comprising a cap coating disposed on the eccentric coating.

7. The coated stent of claim 6 wherein the cap coating is of substantially uniform thickness.

8. The coated stent of claim 6 wherein the cap coating includes a therapeutic agent.

9. The coated stent of claim 6 wherein the cap coating further comprises a first cap portion and a second cap portion, the first cap portion disposed on the first eccentric portion and the second cap portion disposed on the second eccentric portion.

10. The coated stent of claim 9 wherein the second cap portion is thicker than the first cap portion.

11. The coated stent of claim 9 wherein the first cap portion is thicker than the second cap portion.

12. The coated stent of claim 9 wherein the second cap portion is biodegradable.

13. The coated stent of claim 9 wherein the first cap portion includes one therapeutic agent and the second cap portion includes a different therapeutic agent.

14. A method for producing a coated stent comprising:
providing a stent;
mixing a polymer and a therapeutic agent with a solvent to form a polymer/drug solution;
applying the polymer/drug solution to the stent as an eccentric layer; and
curing the eccentric layer to form an eccentric coating.

15. The method of claim 14 wherein the stent has a stent outer diameter, and applying the polymer/drug solution further comprises:
mounting the stent on a coating fixture having a fixture mandrel inside the stent; and
spraying the polymer/drug solution at the stent outer diameter in the direction of the fixture mandrel.

16. The method of claim 14 wherein the stent has a stent outer diameter and a stent inner diameter, and applying the polymer/drug solution further comprises:
applying the polymer/drug solution to the stent inner diameter with an inner pad; and
applying the polymer/drug solution to the stent outer diameter with an outer pad.

17. The method of claim 14 wherein applying the polymer/drug solution to the stent as an eccentric layer further comprises applying the polymer/drug solution by an application method selected from the group consisting of painting, spraying, dipping, wiping, electrostatic deposition, vapor deposition, epitaxial growth, and combinations thereof.

18. The method of claim 14 further comprising:
mixing a polymer with a solvent to form a polymer solution;
applying the polymer solution to the eccentric coating as a cap layer, and
curing the cap layer to form a cap coating.

19. The method of claim 18 wherein the stent has a stent outer diameter and a stent inner diameter, and applying the polymer solution further comprises:
applying the polymer solution to the stent inner diameter with an inner pad; and
applying the polymer solution to the stent outer diameter with an outer pad.

20. The method of claim 18 wherein applying the polymer solution further comprises applying the polymer solution by an application method selected from the group consisting of painting, spraying, dipping, wiping, electrostatic deposition, vapor deposition, epitaxial growth, and combinations thereof.
